(19) United States
(12) Patent Application Publication
(10) Pub. No.: US 2014/0213991 A1
(43) Pub. Date: Jul. 31, 2014

(54) NASAL RELIEF SYSTEM

(71) Applicants: Andrzej Skrzyniarz, Wheeling, IL (US); Magdalena Rajca, Wheeling, IL (US)

(72) Inventors: Andrzej Skrzyniarz, Wheeling, IL (US); Magdalena Rajca, Wheeling, IL (US)

(21) Appl. No.: 13/753,581

(22) Filed: Jan. 30, 2013

(51) Int. Cl. A61M 1/00

(52) U.S. Cl. CPC A61M 1/0031
USPC 604/319

(57) ABSTRACT

A nasal relief system that includes a suction pipe insert with a funnel-shape hollow interior, the first end of the suction pipe insert is releasably inserted into a suction hose of a vacuum cleaner, a flexible nasal hose in communication with the funnel-shape hollow interior of the suction pipe insert and a suction pump in communication with the flexible nasal hose. The system includes a suction regulator that control the suction force from the suction hose of a vacuum cleaner that is set inside the suction pump, a replaceable plastic tip that is inserted into a user's nose to apply the suction force from the flexible nasal hose and a cleaning brush utilized to clean the nasal relief system.

### TECHNICAL FIELD & BACKGROUND

The present invention generally relates to a medical relief system. More specifically, the invention is a nasal relief system.

Many people suffer from allergies, which can cause a stuffy or runny nose. It can be difficult for an individual with rhinitis or other nasal condition to breathe, limiting the enjoyment of regular activities and causing interruptions of sleep.

The present invention is a nasal relief system that safely and effectively reduces irritation, inflammation and cleans out a user's mucous membrane.

### SUMMARY OF THE INVENTION

The present invention is a nasal relief system that safely and effectively reduces irritation, inflammation and cleans out a user's mucous membrane.

It is an object of the present invention to provide a nasal relief system that utilizes a suction pump and a suction regulator to safely and effectively reduce nasal irritation, nasal inflammation and cleans out a user's mucous membrane.

It is an object of the present invention to provide a nasal relief system that also utilizes a replaceable plastic tip and cleaning brush to ensure safety and hygiene.

It is an object of the present invention to provide a nasal relief system that utilizes suction from a vacuum cleaner hose that is reduced and controlled by a suction regulator.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a side view of a nasal relief system, according to an embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is utilized repeatedly. The phrase generally does not refer to the same embodiment, however, it may. The terms “comprising”, “having” and “including” are synonymous, unless the context dictates otherwise.

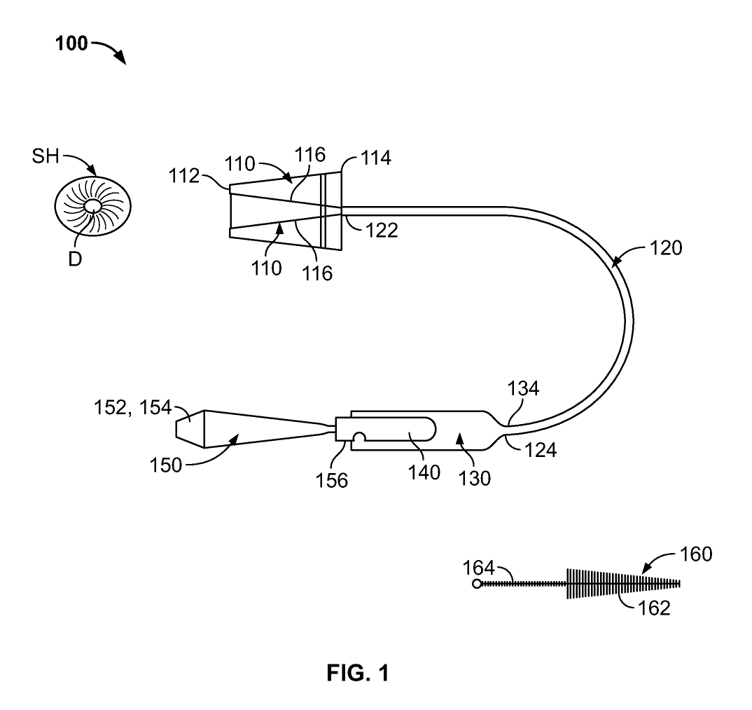

FIG. 1 illustrates a side view of a nasal relief system 100, according to an embodiment of the present invention.

The nasal relief system 100 includes a suction pipe insert 110, a flexible nasal hose 120, a suction pump 130, a suction regulator 140, a replaceable plastic tip 150 and a cleaning brush 160. The suction pipe insert 110 has a first end 112, a second end 114 and a funnel-shape hollow interior 116 with the first end 112 and of the suction pipe insert 110 is releasably inserted into a suction hose of a vacuum cleaner SH. The suction hose of a vacuum cleaner SH provides a suction force to the nasal relief system 100 through the suction pipe insert 110. The suction pipe insert 110 accommodates a plurality of suction hose diameter sizes D and is tapered to ensure a relatively good fit within the suction hose diameter sizes D. The flexible nasal hose 120 has a first end 122 and a second end 124 with the first end 122 of the flexible nasal hose 120 in communication with the second end 114 and the funnel-shape hollow interior 116 of the suction pipe insert 110. The flexible nasal hose 120 is made of clear, flexible plastic but can be made of any other suitable clear flexible material. The suction pump 130 provides a supplemental source of suction to the nasal relief system 100. The suction pump 130 has a first end 132 and a second end 134 with the second end 134 of the suction pump 130 in communication with the second end 124 of the flexible nasal hose 120. The suction regulator 140 is set inside the suction pump 130 and reduces and controls the suction force received from the second end 124 of the flexible nasal hose 120. The replaceable plastic tip 150 has a general cone shape 152 that has a first end 154 and a second end 156. The second end 156 of the replaceable plastic tip 150 receives the suction force from the first end 132 of the suction pump 130. The first end 154 of the replaceable plastic tip 150 is inserted into a user's nose to apply the suction force from the second end 124 of the flexible nasal hose 120 to safely and effectively reduce nasal irritation, nasal inflammation and to clean out a user's mucous membrane. The first end 154 of the replaceable plastic tip 150 is replaced to provide safety and hygiene after each use. The first end 154 of the replaceable plastic tip 150 is made of rubber but can be made of any suitable material. The cleaning brush 160 is utilized to clean the nasal relief system 100. The cleaning brush 160 has an elongated cone-shaped brush head 162 and an elongated thin handle 164 that is fitted to accommodate cleaning the nasal relief system 100.

The nasal relief system is a suction device that is made of clear plastic and has a flexible hose, a pump and a suction regulator that can be relatively quickly and easily attached to a standard household vacuum cleaner hose. Once connected, a uniform suction force from the vacuum cleaner hose is reduced by the suction regulator, ensuring efficacy and safety to clean out a user's mucous membrane. The nasal relief system may be used on an infant, a child or an adult. The nasal relief system features a flexible hose that may be connected to a vacuum cleaner hose to remove nasal secretions and mucous. The nasal relief system is also designed with a replaceable rubber tip to ensure safety and hygiene.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A nasal relief system, comprises:
a suction pipe insert with a first end, a second end and a funnel-shape hollow interior, the first end of the suction pipe insert is releasably inserted into a suction hose of a vacuum cleaner, the suction hose of a vacuum cleaner provides a suction force through the suction pipe insert;
a flexible nasal hose with a first end and a second end, the first end of the flexible nasal hose is in communication with the second end and the funnel-shape hollow interior of the suction pipe insert;
a suction pump with a first end and a second end, the second end of the suction pump is in communication with the second end of the flexible nasal hose, the suction pump provides a supplemental source of suction to the nasal relief system;
a suction regulator that is set inside the suction pump, the suction regulator reduces and controls the suction force received from the second end of the flexible nasal hose;
a replaceable plastic tip with a first end, a second end and a general cone-shape, the second end of the replaceable plastic tip receives the suction force from the first end of the suction pump, the first end of the replaceable plastic tip is inserted into a user's nose to apply the suction force from the second end of the flexible nasal hose; and
a cleaning brush utilized to clean the nasal relief system.

2. The nasal relief system according to claim 1, wherein the suction pipe insert accommodates a plurality of suction hose diameter sizes.

3. The nasal relief system according to claim 2, wherein the suction pipe is tapered to ensure a good fit within the suction hose diameter sizes.

4. The nasal relief system according to claim 1, wherein the suction force from the second end of the flexible nasal hose safely and effectively reduces nasal irritation, nasal inflammation and cleans out a user's mucous membrane.

5. The nasal relief system according to claim 1, wherein the first end of the replaceable plastic tip is replaced to provide safety and hygiene after each use.

6. The nasal relief system according to claim 1, the cleaning brush is fitted to accommodate cleaning the nasal relief system.

7. The nasal relief system according to claim 1, wherein the cleaning brush has an elongated cone-shaped brush head.

8. A nasal relief system, comprises:
a suction pipe insert with a first end, a second end and a funnel-shape hollow interior, the first end of the suction pipe insert is releasably inserted into a suction hose of a vacuum cleaner, the suction hose of a vacuum cleaner provides a suction force through the suction pipe insert;
a flexible nasal hose with a first end and a second end, the first end of the flexible nasal hose is in communication with the second end and the funnel-shape hollow interior of the suction pipe insert;
a suction pump with a first end and a second end, the second end of the suction pump is in communication with the second end of the flexible nasal hose, the suction pump provides a supplemental source of suction to the nasal relief system;
a suction regulator that is set inside the suction pump, the suction regulator reduces and controls the suction force received from the second end of the flexible nasal hose;
a replaceable plastic tip with a first end, a second end and a general cone-shape, the second end of the replaceable plastic tip receives the suction force from the first end of the suction pump, the first end of the replaceable plastic tip is inserted into a user's nose to apply the suction force from the second end of the flexible nasal hose and the first end of the replaceable plastic tip is replaced to provide safety and hygiene after each use; and
a cleaning brush utilized to clean the nasal relief system.

9. The nasal relief system according to claim 8, wherein the suction pipe insert accommodates a plurality of suction hose diameter sizes.

10. The nasal relief system according to claim 9, wherein the suction pipe is tapered to ensure a good fit within the suction hose diameter sizes.

11. The nasal relief system according to claim 8, wherein the suction force from the second end of the flexible nasal hose safely and effectively reduces nasal irritation, nasal inflammation and cleans out a user's mucous membrane.

12. The nasal relief system according to claim 8, the cleaning brush is fitted to accommodate cleaning the nasal relief system.

13. The nasal relief system according to claim 8, wherein the cleaning brush has an elongated cone-shaped brush head.

14. The nasal relief system according to claim 8, wherein the cleaning brush has an elongated thin handle.

15. A nasal relief system, comprises:
a suction pipe insert with a first end, a second end and a funnel-shape hollow interior, the first end of the suction pipe insert is releasably inserted into a suction hose of a vacuum cleaner, the suction hose of a vacuum cleaner provides a suction force through the suction pipe insert;
a flexible nasal hose with a first end and a second end, the first end of the flexible nasal hose is in communication with the second end and the funnel-shape hollow interior of the suction pipe insert, the suction force from the second end of the flexible nasal hose safely and effectively reduces nasal irritation, nasal inflammation and cleans out a user's mucous membrane;
a suction pump with a first end and a second end, the second end of the suction pump is in communication with the second end of the flexible nasal hose, the suction pump provides a supplemental source of suction to the nasal relief system;
a suction regulator that is set inside the suction pump, the suction regulator reduces and controls the suction force received from the second end of the flexible nasal hose;
a replaceable plastic tip with a first end, a second end and a general cone-shape, the second end of the replaceable plastic tip receives the suction force from the first end of the suction pump, the first end of the replaceable plastic tip is inserted into a user's nose to apply the suction force from the second end of the flexible nasal hose and the first end of the replaceable plastic tip is replaced to provide safety and hygiene after each use; and
a cleaning brush utilized to clean the nasal relief system, the cleaning brush is fitted to accommodate cleaning the nasal relief system and the cleaning brush has an elongated thin handle.

16. The nasal relief system according to claim 15, wherein the suction pipe insert accommodates a plurality of suction hose diameter sizes.

17. The nasal relief system according to claim 16, wherein the suction pipe is tapered to ensure a good fit within the suction hose diameter sizes.

18. The nasal relief system according to claim 15, wherein the cleaning brush has an elongated cone-shaped brush head.

19. The nasal relief system according to claim 15, wherein the nasal hose is made of clear, flexible plastic.

20. The nasal relief system according to claim 15, wherein the first end of the replaceable plastic tip is made of rubber.
